(19) United States
(12) Patent Application Publication
(10) Pub. No.: US 2021/0100786 A1
(43) Pub. Date: Apr. 8, 2021

(54) Pharmaceutical Formulations

(71) Applicant: CIPLA LIMITED, Mumbai (IN)

(72) Inventors: Geena MALHOTRA, Mumbai (IN); Kalpana JOSHI, Thane (IN); Preeti RAUT, Mumbai (IN); Jeevan GHOSALKAR, Thane (West) (IN); Neeta DIXIT, Thane (West) (IN)

(21) Appl. No.: 17/048,303

(22) PCT Filed: Apr. 5, 2019
(86) PCT No.: PCT/IN2019/050281

§ 371 Date: Oct. 16, 2020

(30) Foreign Application Priority Data
Apr. 5, 2018 (IN) 201821013065

(51) Int. Cl. A61K 31/47; A61K 9/28; A61K 9/20; A61K 9/00; A61K 31/424

(52) U.S. Cl. CPC A61K 31/47; A61K 9/282; A61K 9/2054; A61K 31/424; A61K 9/2013; A61K 9/2009; A61K 9/0056; A61K 9/2059

(57) ABSTRACT

A pharmaceutical formulation is provided comprising combination of anti-tuberculosis drug drugs optionally in combination of bioenhancers. The formulation is used for the treatment of diseases caused by mycobacterium tuberculosis. The process of preparation of the formulation is also provided.

### CROSS-REFERENCE TO RELATED APPLICATION

This Application is a filing under 35 U.S.C. 371 of International Application No. PCT/IN2019/050281 filed Apr. 5, 2019, entitled “Pharmaceutical Formulations,” which claims the benefit of Indian Application 201821013065, filed Apr. 5, 2018, the contents of which are hereby incorporated by reference in their entirety.

### FIELD OF INVENTION

The present invention relates to pharmaceutical formulation comprising at least one new antituberculosis agent and at least one bioenhancer and optionally at least one pharmaceutically acceptable excipients. The present invention also provides manufacturing processes thereof and use of the said composition for prevention, treatment or prophylaxis of diseases in the patients in need thereof.

### BACKGROUND AND PRIOR ART

Tuberculosis (TB) remains a major health problem worldwide and continues to be a significant cause of mortality and morbidity worldwide. Tuberculosis (TB) affects one-third of the world's population, with 10.4 million new cases and 1.8 million deaths reported in 2015. The infectious agent, Mycobacterium tuberculosis, is a deadly infectious pathogen causing tuberculosis (TB) worldwide, which can be transmitted by aerosols from a contaminated individual, has a unique ability to survive within the host, alternating between active and latent disease states, and escaping the immune system defenses.

Although TB can be cured by optimum chemotherapy, but the emergence of drug resistant tuberculosis [such as multidrug-resistant tuberculosis (MDR-TB), extensively drug-resistant tuberculosis (XDRTB) and totally drug resistant tuberculosis (TDR-TB)] has created a new challenge to combat the adverse situation of the disease. Between 5 and 10% of people with HIV are also infected with tuberculosis (often called co-infection). According to WHO (2016) 10.4 million new cases with 1.5 million deaths including 0.4 million individuals with HIV-TB co-infection were reported globally. Also, since among 10.4 million cases, 480,000 affected by multidrug-resistant tuberculosis (MDR-TB)-10% meeting the criteria for extensively drug-resistant tuberculosis (XDR-TB) and 100,000 by rifampicin-resistant TB and 190,000 deaths, thus the disease is a public health priority. Due to various complexities and high burden of HIV-TB co-infection, treatments of MDR-TB, XDRTB, and TDR-TB are problematic. Clearly, currently available drugs and vaccines have had no significant impact on TB control. Until today, XDRTB has no guidelines or evidence that may guide its treatment, showing a cure rate of only 26% using second and third line drugs. Unfortunately, the growing burden of antibiotic resistance is coupled with decreased effort in the development of new antibiotics.

The spread of drug resistant TB is a major threat to global TB control. These strains are now entrenched in most countries and are spreading at an alarming rate. Multi-drug resistant (MDR) TB isolates are resistant to isoniazid (INH) and rifampicin, the two frontline drugs for TB treatment, and have been detected in every country surveyed. Hundreds of thousands of people worldwide are going untreated and continuing to spread drug resistant forms of the disease. Extensively drug-resistant (XDR) TB strains, first detected in 2006, are resistant to front-line and second-line anti-tubercular anti-biotics. XDR-TB is now present in over 100 countries and represents approximately 10% of MDR-TB cases. The disease is currently treated with a standard therapy as combination of four anti-microbial drugs isoniazid, rifampicin, pyrazinamide, and ethambutol, during a six months course that does not favour patient compliance. Thus, TB treatment is long; standard treatment for drug sensitive strains is 6 to 12 months, while patients with drug resistant TB must endure a longer course of treatment (24 months or longer) with harsh side effects, high cost and a low chance of cure. Delayed diagnosis and inappropriate treatment leads to multiplication of resistance; this is best highlighted by the alarming emergence of totally drug resistant (TDR) TB, which is essentially untreatable using current drugs. The combination of long treatment and side effects results in poor compliance, which is a major contributor to the development of resistance. Thus, it is evident that current methods of treatment and control for TB are not sustainable in the face of highly drug resistant TB.

Resistance in Mycobacterium tuberculosis is mainly due to the occurrence of spontaneous mutations and followed by selection of mutants by subsequent treatment. However, some resistant clinical isolates do not present mutations in any genes associated with resistance to a given antibiotic, which suggests that other mechanism(s) are involved in the development of drug resistance, namely the presence of efflux pump systems. This mechanism of resistance results in efflux of a variety of anti-TB drugs from the bacterial cell, thereby decreasing the intracellular drug concentration. In doing so, the bacillus is able to render the antibiotic treatment ineffective.

Recently, to overcome the resistance and for effective treatment of resistant Mycobacterium tuberculosis strains, new TB drugs such as bedaquiline, delamanid, pretomanid and the like are developed. The new TB drugs are increasingly used to treat multidrug-resistant (MDR-) and extensively drug-resistant tuberculosis (XDR-TB). The new regimen proposed for treatment of XDR tuberculosis comprises administration of combination of new anti-tuberculosis drugs with at least 4 other drugs to which the patient's MDR-TB isolate is likely to be susceptible. However, there always remains a risk for interactions among the drugs or with other non-antituberculosis drugs. One way to reduce the interactions among drugs, lower the dose, reduce the chances of developing resistance for the drugs, reducing the side effects and yet achieving the desired therapeutic effect is by increasing the bioavailability of drugs. To improve the bioavailability and to boost the effectiveness of anti-tuberculosis drugs, bioenhancers are used in combination with the anti-tuberculosis drugs.

Hence there is an obvious and urgent need to develop formulations wherein the drug characteristics of new antituberculosis drugs is improved by improving bioavailability of the drugs which will be eventually effective against drug resistant Mycobacterium tuberculosis strains, as this will further reduce the dose and duration of treatment regimens and lead to patient compliance.

Bioenhancers specifically cause inhibition of the cytochrome P450 3A4 enzyme system and efflux pump inhibition leading to an increase in the plasma concentrations of the co-administered antituberculosis drugs. Efflux pumps are membrane proteins involved in the transport of a variety of substrates, including drugs, from the interior to the exterior of the cell. Thus, efflux pumps extrude the drug to the exterior of the cell, preventing access to its target. These transporters are mainly responsible for intestinal permeability thereby predicting the bioavailability of a drug. In addition, they are also present on Gram-positive and -negative bacteria. The intrinsic resistance of mycobacteria to most drugs is mainly attributed to the synergy between their relatively impermeable cell wall and efflux systems. Thus, when a bioenhancer is co-administered with the antituberculosis drug, it interferes with the transport of anti-tuberculosis drug from the interior to the exterior of the cell, which causes the anti-tuberculosis drug to remain in the body for a longer period and at a higher concentration.

Therefore, there remains a need to provide a new combination therapy of some anti-tuberculosis drugs or combination therapy of anti-tuberculosis drugs with bioenhancer for the treatment of tuberculosis and HIV which reduces the dose of such anti-tuberculosis drugs, side effects exhibited by these drugs as well as maintains the optimal concentration of the same. Further, use of a bioenhancer would eliminate or reduce interactions with other anti-tuberculosis and non-anti-tuberculosis medications that would be concurrently administered

There are many drugs which when co-administered with antituberculosis drugs can act as bioenhancers or efflux pump inhibitors.

U.S. Pat. No. 5,439,891 discloses a pharmaceutical composition for the treatment of tuberculosis and leprosy comprising piperine in combination with known antituberculosis or antileprosy drugs or the mixtures thereof.

WO2011012987 discloses solid oral pharmaceutical composition comprising Rifampicin, Pipeline and Isoniazid wherein the bioavailability of Rifampicin is enhanced in presence of isoniazid.

None of the prior arts specifically disclose the use of bioenhancer to enhance the bioavailability of new antituberculosis drugs. Therefore, there remains a need to provide a combination therapy of an bioenhancer with new-antituberculosis drugs for the treatment of (MDR) TB, (XDR) TB, (TDR) TB which reduces the dose of antituberculosis drugs, improve efficacy and tolerance, reduce the resistance to drugs, reduce side effects exhibited by these drugs as well as maintains the optimal concentration of the same. Thus, the combination of antituberculosis drug with bioenhancer will have overall effect on bioavailability, decreased resistance, and improvement in the toxicity profile. These efflux pump inhibitors might reduce the cost of antituberculosis therapy, reduce pill burden for patients, and/or reduce the risk of sub therapeutic antituberculosis concentrations (e.g., development of resistance as well as enhance adherence to antituberculosis therapy) and thus improve patient compliance.

Thus, looking at the rapid increase in resistance of bacterium against the old antituberculosis drugs and considering the unmet medical needs, the inventors of present invention have developed pharmaceutical formulation comprising at least one new antituberculosis agent and at least one bioenhancer.

### OBJECT OF THE INVENTION

An object of the present invention is to provide a pharmaceutical formulation comprising at least one new anti-tuberculosis drug and at least one bioenhancer.

Another object of the present invention is to provide a pharmaceutical formulation comprising at least one new anti-tuberculosis drug and at least one bioenhancer with reduced side effects.

Yet another object of the present invention is to provide a pharmaceutical formulation comprising at least one new anti-tuberculosis drug and at least one bioenhancer with reduced drug interactions.

Another object of the present invention is to provide a pharmaceutical formulation comprising new anti-tuberculosis drug and at least one bioenhancer for once or twice a day administration.

Yet another object of the present invention is to provide a pharmaceutical formulation comprising new anti-tuberculosis drug and at least one bioenhancer in the form of a kit.

Yet another object of the present invention is to provide a method of prevention, treatment or prophylaxis of diseases caused by Mycobacterium tuberculosis, which method comprises administering at least one new anti-tuberculosis drug and at least one bioenhancer.

Yet another object of the present invention is to provide the use of a pharmaceutical formulation comprising at least one new anti-tuberculosis drug and at least one bioenhancer for the treatment or prophylaxis of (MDR) TB, (XDR) TB, (TDR) TB caused by Mycobacterium tuberculosis.

### SUMMARY OF THE INVENTION

According to an aspect of the present invention, there is provided a pharmaceutical formulation comprising at least one new anti-tuberculosis drug and at least one bioenhancer and one or more pharmaceutically acceptable excipients.

According to another aspect of the invention, there is provided a process for preparing a pharmaceutical formulation comprising at least one new anti-tuberculosis drug and at least one bioenhancer with at least one or more pharmaceutically acceptable excipients.

According to another aspect of the present invention there is provided a method of treating (MDR)TB, (XDR)TB, (TDR)TB caused by Mycobacterium tuberculosis, such method comprising administering a therapeutically effective amount of a pharmaceutical formulation comprising at least one new anti-tuberculosis drug and at least one bioenhancer according to the present invention to a patient in need thereof.

According to another aspect of the present invention there is provided the use of a pharmaceutical formulation comprising one new anti-tuberculosis drug and at least one bioenhancer according to the present invention in the manufacture of a medicament for the treatment of (MDR) TB, (XDR) TB, (TDR) TB caused by Mycobacterium tuberculosis.

### DETAILED DESCRIPTION OF THE INVENTION

For the treatment of diseases caused by Mycobacterium tuberculosis it is essential that maximum amount of the drug reaches the site of action. Most new anti-tuberculosis drugs either have poor solubility and/or poor permeability which deteriorates the bioavailability of the drug to a major extent.

The inventors of the present invention have found ways to address the bioavailability problems of new anti-tuberculosis drugs. In particular, the inventors have found that, the bioavailability properties of these class of drugs can be improved by using bioenhancer.

The present invention relates to a pharmaceutical formulation having increased therapeutic efficacy. The formulation of the present invention is particularly useful for the treatment of (MDR)TB, (XDR)TB, (TDR)TB caused by Mycobacterium tuberculosis, and co-infection of HIV and TB.

The term “antituberculosis drugs” and “bioenhancer” is used in broad sense to include not only the “antituberculosis” per se and “bioenhancer” per se but also its pharmaceutically acceptable derivatives thereof. Suitable pharmaceutically acceptable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

The new anti-tuberculosis drugs, according to the present invention, include, but are not limited to Bedaquiline, Delamanid, Pretomanid, Sutezolid and any combinations thereof. Preferably, the new antituberculosis drug, according to the present invention is bedaquiline or its pharmaceutically acceptable salt or its derivative thereof. In an embodiment, preferably the new antituberculosis drug, according to the present invention is delamanid or its pharmaceutically acceptable salt or its derivative thereof.

Bedaquiline, new anti-TB drug, trade name Sirturo, approved in December 2012 by USFDA. The development of Bedaquiline is significant because it was the first TB antibiotic approved for the pharmaceutical market in forty years, and it is particularly effective for treating MDR-TB cases. It is metabolized by CYP3A4 to N-monodes methyl metabolite, which is 4-6 times less potent than the parent drug. Most importantly, alarmingly, efflux-mediated bedaquiline resistance, as well as efflux-mediated cross-resistance to clofazimine, has been identified in treatment failures. Thus, this mechanism of resistance results in efflux of anti-TB drugs from the bacterial cell and may render the antibiotic treatment ineffective. The recommended dosage of SIRTURO is 2-4 tablets of 100 mg taken once daily with food.

Delamanid, also known by its trade name of Deltyba, is the first in a new class of TB drugs called nitroimidazoles. It is available as 50 mg tablets and the recommended dose is needs to be taken for six months. The amount of bedaquiline or its pharmaceutically acceptable salts or derivatives present in the pharmaceutical formulation is from about 1% to about 50% w/w of the total formulation, preferably from about 10% to about 40% w/w of the total formulation.

The formulation of present invention comprises at least one anti-tuberculosis drugs and at least one bioenhancer and optionally one pharmaceutically acceptable excipient. In an embodiment, the formulation of present invention comprises bedaquiline or its pharmaceutically acceptable salts or derivative thereof and at least one bioenhancer or its derivatives thereof and optionally one pharmaceutically acceptable excipient. Preferably, the dose of bedaquiline or its pharmaceutically acceptable salt according to the present invention ranges from about 20 mg to 200 mg for once, twice or thrice a day.

In another embodiment, the formulation of present invention comprises delamanid or its pharmaceutically acceptable salts or derivative thereof and at least one bioenhancer and optionally one pharmaceutically acceptable excipient. Preferably, the dose of delamanid its pharmaceutically acceptable salt, according to the present invention, ranges from about 10 mg to 100 mg for once, twice or thrice a day.

The pharmaceutical formulation of present invention further comprises of at least one bioenhancer Bioenhancers or bioavailability enhancers' are drug facilitators and the molecules which by themselves do not show typical pharmacological activity but when used in combination they enhance the activity of drug molecule in several ways including increasing bioavailability of the drug across the membrane, potentiating the drug molecule by conformational interaction, acting as receptors for drug molecule and making target cells more receptive to drugs. These are also termed as ‘absorption enhancers’ which are functional excipients included in formulations to improve the absorption of a pharmacologically active drug. Bioenhancers act by various mechanisms of action such as DNA receptor binding, modulation of cell signal transduction and inhibition of drug efflux pump, inhibition of human P-glycoprotein and cytochrome P450 3A4 and the like.

The bioenhancers according to the present invention, include, but are not limited to, piperine, garlic, Carum carvi, Currinum cyrrinurn lysergol, naringin, quercetin, niaziridin, glycyrrhizin, stevia, cow urine, distillate ginger, or any combination thereof. The term “bioenhancer”, according to present invention, is preferably an alkaloid. More preferably, the bioenhancers/efflux pump inhibitor/pharmacokinetic booster or enhancer is piperine, isopiperine, tetrahydropiperine, chavicine, isochavicine and their analogs or derivatives thereof.

The compound piperine may be obtained as an extract from the fruit of piper nigrum. The fruit of black pepper (Piper nigrum L.) and long pepper (Piper longum L.) are both important medicinal herbs Black pepper contains approximately 5-9% piperine and is listed by the FDA as an herb which is generally recognized as safe (GRAS) for its intended use as spice, seasoning, or flavoring. The extract from black pepper has higher concentration of piperine than natural black pepper and extract from piper longum having a higher concentration of piperine than natural piper longum.

Piperine is chemically known as (1-2E, 4E-piperinoyl-piperidine) and is structurally represented as below

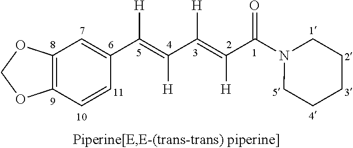 [SMILES: O=C(/C=C/C=C/c1ccc2c(c1)OCO2)N1CCCCC1]

Without being bound to any theory whatsoever, Piperine may increase the drug bioavailability by inhibiting enzymes which participate in the biotransformation of drugs and thus preventing their inactivation and elimination. It also inhibits p-glycoprotein and major drug metabolizing enzyme CYP3A4. the ‘pump’ protein that removes substances from cells and can decrease the intestinal production of glucuronic acid, thereby permitting more substances to enter the body in active form.

Piperine may enhance the drug bioavailability by promoting rapid absorption of drugs and nutrients by increasing blood supply to the gastrointestinal tract, decreasing hydrochloric acid secretion to prevent the breakdown of some drugs, increasing the emulsifying content of the gut, increasing enzymes like γ-glutamyl transpeptidase which participate in active and passive transport of nutrients to the intestinal cells.

Piperine has also been reported to occur in other Piper species i.e. P. acutisleginum, album, argyrophylum, attenuatum, aurantiacum, betle, callosum, chaba, cubeba, guineense, hancei, khasiana, longum, macropodum, nepalense, novae hollandiae, peepuloides, retrokacturn, sylvaticum.

Tetrahydro piperine is a structural analog of Piperine. The two double bonds at position 2 and 4 are saturated to give a tetrahydro analog. Tetrahydropiperine is chemically known as 5-(1,3-benzodioxol-5-yl)-1-piperidin-1-ylpentan-1-one and is structurally represented as below.

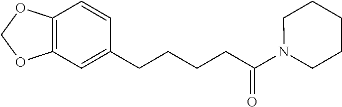 [SMILES: O=C(CCCCc1ccc2c(c1)OCO2)N1CCCCC1]

Tetrahydropiperine occurs like piperine naturally in black pepper (about 0.7% in black pepper oleoresin). Tetrahydropiperine can be synthesized from piperine which is previously extracted from black pepper oleoresin.

The term “analogs or derivatives” of tetrahydropiperine is used in broad sense to include alkyltetrahydropiperines, such as methyltetrahydropiperine or ethyltetrahydropiperine, dialkyltetrahydropiperines, such as dimethyltetrahydropiperine or diethyltetrahydropiperine, alkoxylated tetrahydropiperine, such as methoxy tetrahydropiperine, hydroxylated tetrahydropiperine, e.g. 1-[(5,3-benzo di oxyl-5-yl)-1-hydroxy-2,4-pentadienyl]-piperine, 1-[(5,3-b enzodioxyl-5-yl)-1-methoxy-2,4-pentadienyl]-piperine, halogenated tetrahydropiperine, such as 1-[(5,3-benzodioxyl-5-yl)-1-oxo-4-halo-2-pentenyl]-piperine and 1-[(5, 3-benzo dioxyl-5-yl)-1-oxo-2-halo-4-pentenyl]-piperine, dihydropiperine, alkyldihydropiperines, such as methyldihydropiperine or ethyldihydropiperine, dialkyldihydropiperines, such as. dimethyldihydropiperine or diethyldihydropiperine, alkoxylated dihydropiperine such as methoxy dihydropiperine, and halogenated dihydropiperine and their pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

In an embodiment of present invention, the piperine used in the present invention may be occurring naturally in the fruits or may be prepared synthetically by the process well known in the art. The piperine or its derivatives prepared synthetically or extracted from the naturally occurring fruits are substantially pure. The term “substantially pure piperine” herein refers to piperine having purity (measured by HPLC) above 99.5%, preferably above about 99.7%, and more preferably above about 99.9%.

The bioenhancing dose of piperine as used in the present invention is a maximum of approximately 15 mg/person/day, or no more than 20 mg/day in divided doses, which corresponds to from several thousands to up to 40,000 times less than the LD50 dose of piperine, as established in various experiments on rodents.

Preferably, according to present invention, the dose of piperine ranges from about 0.5 mg to about 400 mg, and the dose of tetrahydropiperine ranges from about 0.5 mg to about 400 mg.

In one embodiment, the dose of the piperine and/or the tetrahydropiperine ranges from about 0.5 mg, 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, to about 400 mg.

In another embodiment, the ratio of the antituberculosis drug to the bioenhancer is from about 100:1 to about 1:1 by weight.

Preferably, the present invention provides a pharmaceutical formulation comprising bedaquiline or its pharmaceutically acceptable salt in combination with piperine or its derivative and at least one pharmaceutically acceptable excipient. In an embodiment, present invention thus provides a pharmaceutical formulation comprising bedaquiline or its pharmaceutically acceptable salt in combination with piperine or its derivative for once, twice or thrice a day administration.

In another embodiment, the present invention provides a pharmaceutical formulation comprising delamanid in combination with piperine and at least one pharmaceutically acceptable excipient.

The pharmaceutical formulations of the present invention comprise at least one new antituberculosis drug and piperine and its analogs or derivatives. These active ingredients are formulated for simultaneous, separate or sequential administration. When the active ingredients are administered sequentially, either the at least one new antituberculosis drug or piperine, may be administered first. When administration is simultaneous, the active ingredients may be administered either in the same or different pharmaceutical compositions. Adjunctive therapy, i.e. where one active ingredient is used as the primary treatment and the other active ingredient(s) is/are used to assist that primary treatment is also an embodiment of the present invention.

The pharmaceutical formulation of present invention comprising at least one new antituberculosis drug and at least one bioenhancer, further comprises of additional old tuberculosis drugs such as Isoniazid, Rifampicin, Pyrazinamide, Ethambutol, and Streptomycin.

The pharmaceutical formulation of present invention comprising at least one new antituberculosis drug and at least one bioenhancer, further comprises of additional anti-HIV drugs when the treatment is aimed at co-infection of HIV and tuberculosis. Such additional anti-HIV drugs are HIV reverse transcriptase inhibitors (nucleoside and non-nucleoside inhibitors), protease inhibitors, entry inhibitors (also known as fusion inhibitors), integrase inhibitors and viral DNA polymerase inhibitors such as, but not limited to, zidovudine or AZT, didanosine, stavudine, lamivudine, zalcitabine, tenofovir disoproxil fumarate, tenofovir alafenamide, emtricitabine, efavirenz, doravarine, lamivudine, zidovudine, didanosine, stavudine, abacavir, etravirine, delavirdine, nevirapine or their salt, solvate, esters, derivatives, hydrate, enantiomer, polymorph prodrugs, tautomers, isomers, anhydrates or mixtures thereof.

The inventors of the present invention have also found that the bioavailability properties of the new antituberculosis drug may also be improved by nanosizing. Preferably, the pharmaceutical formulation of present invention comprises at least one nanosized new antituberculosis drug and at least one bioenhancer. The pharmaceutical formulation of present invention may also comprise of at least one new antituberculosis drug and at least one nano sized bioenhancer. The pharmaceutical formulation of present invention may also further comprise of at least one nanosized new antituberculosis drug and at least one nano sized bioenhancer. In one embodiment, the pharmaceutical composition is administered via nanoparticles having a size of about 1 nanometer (nm) to about 50 nm.

The term “pharmaceutical composition” includes dosage forms such as but not limited to, unit dosage forms including tablets, capsules (filled with powders,pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, minitablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Preferably, the mini-tablets or granules filled in such hard gelatin capsules or sachets are directly administered or by sprinkling the mini-tablet or granules on regular meals. Alternatively, the mini-tablets or granules filled in hard gelatin capsules or sachets may be administered with liquid or semi-solid beverages such as but not limited to, juices, water.

The mini-tablets or granules, according to the present invention, may also optionally be coated. Preferably, mini-tablets or granules, according to the present invention, may be film coated. More preferably, the mini-tablets or granules may be seal coated and then film coated and further filled in hard gelatin capsules or sachets. It is further well known in the art that a tablet formulation is the preferred solid dosage form due to its greater stability, less risk of chemical interaction between different medicaments, smaller bulk, accurate dosage, and ease of production.

Solid unit dosage forms, according to the present invention, are preferably in the form of tablets either single or bilayered or multilayered tablets but other conventional dosages such as powders, pellets, capsules and sachets may fall within the scope of this invention.

According to a further embodiment of the invention, there is provided a pharmaceutical formulation comprising at least one new antituberculosis drug and piperine as a combined preparation for simultaneous, separate or sequential use for treatment of (MDR) TB, (XDR) TB, (TDR) TB caused by Mycobacterium tuberculosis. When administration is simultaneous, the active ingredients may be administered either in the same or different pharmaceutical compositions. Adjunctive therapy, i.e. where one active ingredient is used as the primary treatment and the other active ingredient(s) is/are used to assist that primary treatment is also an embodiment of the present invention.

Accordingly, there is provided a pharmaceutical formulation comprising bedaquiline or its pharmaceutically acceptable salt and piperine and/or tetrahydropiperine or any of its derivatives as a combined preparation for simultaneous, separate or sequential use for treatment of diseases (MDR) TB, (XDR) TB, (TDR) TB caused by Mycobacterium tuberculosis.

There is also provided a pharmaceutical formulation comprising delamanid or its pharmaceutically acceptable salt and piperine and/or tetrahydropiperine or any of its derivatives as a combined preparation for simultaneous, separate or sequential use for treatment of diseases (MDR) TB, (XDR) TB, (TDR) TB caused by Mycobacterium tuberculosis.

According to another embodiment, the pharmaceutical formulation may be administered as a single layered or bilayererd or multilayered tablet wherein each layer may or may not contain drug/drugs along with pharmaceutically acceptable excipients which are then compressed to provide either a single layered, bilayered or multilayered tablet.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The pharmaceutical formulation of present invention can be prepared by conventional processes known in the art using commonly available equipment such as direct compression, wet granulation, and are not intended to limit the scope of the invention to form the desired dosage form.

Accordingly, when the pharmaceutical composition is provided in unit dosage forms, as discussed above, the unit dosage form can be uncoated or coated.

The present invention provides a pharmaceutical formulation comprising anti-tuberculosis drug or pharmaceutically acceptable salts, derivatives thereof and piperine or its derivatives thereof so that the bioavailability of tuberculosis drug is increased. According to embodiment of present invention, there is provided a method of increasing bioavailability from about 10% to about 100% of bedaquiline by providing formulation comprising bedaquiline or pharmaceutically acceptable salts, derivatives thereof and piperine or its derivatives thereof such that the method comprises administering a therapeutically effective amount of bedaquiline or its pharmaceutically acceptable salts, derivatives thereof and piperine or its derivative thereof as a combination product simultaneously, separately or sequentially to a patient in need thereof.

According to another embodiment of present invention, a method of decreasing the dose of bedaquiline from about from about 5% to about 95% is provided wherein the method comprising administering therapeutically effective amount of bedaquiline or its pharmaceutically acceptable salts or derivatives thereof, piperine or its pharmaceutically acceptable derivatives thereof as a combination product simultaneously, separately or sequentially to a patient in need thereof.

A kit comprising therapeutically effective amount of bedaquiline or its pharmaceutically acceptable salts or derivatives thereof in an amount effective and piperine or its pharmaceutically acceptable derivative thereof to treat diseases caused by mycobacterium tuberculosis. One embodiment of present invention is a kit wherein the bedaquiline or its pharmaceutically acceptable salts or derivatives thereof; piperine or its pharmaceutically acceptable derivative thereof are present in same or separate formulation for simultaneously, separately or sequentially to a patient in need thereof.

The present invention also provides a method of treating diseases caused by mycobacterium tuberculosis, especially (MDR) TB, (XDR) TB, (TDR) TB, such method comprising administering a therapeutically effective amount of a pharmaceutical formulation comprising at least one new antituberculosis drug and at least one bioenhancer to a patient in need thereof.

In another embodiment of present invention, a method of treating diseases caused by mycobacterium tuberculosis in a patient in need of treatment thereof, the method comprising administering a pharmaceutical composition comprising a therapeutically effective amount of bedaquiline or its pharmaceutically acceptable salts or derivatives thereof; piperine or its pharmaceutically acceptable derivative thereof; and optionally one or more pharmaceutically acceptable excipients.

The present invention also provides use of a pharmaceutical composition comprising antituberculosis drug such as bedaquiline, delamanid and piperine or its derivative thereof according to the present invention in the manufacture of a medicament for the treatment of (MDR) TB, (XDR) TB, (TDR) TB caused by Mycobacterium tuberculosis.

These and other aspects of the present application will be further appreciated upon consideration of the following examples, which are intended to illustrate certain particular embodiments of the application but are not intended to limit its scope, as defined by the claims.

### EXAMPLES

### Example 1

Bedaquiline and Piperine film coated tablets

TABLE 1

| Ingredient | Quantity (%) |
|---|---|
| Blending | |
| Bedaquiline fumarate | 30.22 |
| Piperine | 5.00 |
| Microcrystalline | 12.52 |
| cellulose | |
| Lactose monohydrate | 36.25 |
| Croscarmellose sodium | 3.00 |
| Corn starch | 5.00 |
| Binder | |
| Hypromellose | 3.00 |
| Polysorbate 20 | 0.50 |
| Purified water | — |
| Blending and | |
| lubrication | |
| Croscarmellose sodium | 3.00 |
| Colloidal silicon dioxide | 0.50 |
| Magnesium Stearate | 1.00 |
| Total weight of tablet | 100.00 |

Manufacturing Procedure:
1. Bedaquiline fumarate, piperine, microcrystalline cellulose, lactose monohydrate Croscarmellose sodium and Corn starch were weighed, sifted and blended.
2. Hypromellose and polysorbate 20 were added to purified water until dissolved.
3. The blend of step 1 was granulated with solution of step 2.
4. The granules of step 3 were granulated to suitable size.
5. Croscarmellose sodium, colloidal silicon dioxide and magnesium stearate were blended and added with granules of step 3.
6. The blend obtained in step (5) was compressed to prepare tablets.

### Example 2

Bedaquiline and Piperine capsules

TABLE 2

| Ingredient | Quantity (%) |
|---|---|
| Blending | |
| Bedaquiline fumarate | 30.22 |
| Piperine | 5.00 |
| Microcrystalline | 12.52 |
| cellulose | |
| Lactose monohydrate | 36.25 |
| Croscarmellose sodium | 3.00 |
| Corn starch | 5.00 |
| Binder | |
| Hypromellose | 3.00 |
| Polysorbate 20 | 0.50 |
| Purified water | — |
| Blending and | |
| lubrication | |
| Croscarmellose sodium | 3.00 |
| Colloidal silicon dioxide | 0.50 |
| Magnesium Stearate | 1.00 |
| Total fill weight | 100.00 |
| Capsule filling | |
| Empty hard gelatine | 95.00 |
| capsules shell size 0 | |
| Total weight of capsule | — |

Manufacturing Procedure:
1. Bedaquiline fumarate, piperine, microcrystalline cellulose, lactose monohydrate Croscarmellose sodium and Corn starch were weighed, sifted and blended.
2. Hypromellose and polysorbate 20 were added to purified water until dissolved.
3. The blend of step 1 was granulated with solution of step 2.
4. The granules of step 3 were granulated to suitable size.
5. Croscarmellose sodium, colloidal silicon dioxide and magnesium stearate were blended and added with granules of step 3.
6. Fill this blend on a suitable Capsule filling machine to prepare capsules.

### Example 3

Bedaquiline and Piperine oral disintegrating tablets

TABLE 3

| Ingredient | Quantity (%) |
|---|---|
| Blending | |
| Bedaquiline fumarate | 30.22 |
| Piperine | 5.00 |
| Microcrystalline | 12.52 |
| cellulose | |
| Lactose monohydrate | 36.25 |
| Croscarmellose sodium | 3.00 |
| Corn starch | 5.00 |
| Binder | |
| Hypromellose | 3.00 |
| Polysorbate 20 | 0.50 |
| Purified water | — |
| Blending | |
| Crospovidone NF | 3.75 |
| Aspartame NF | 0.75 |
| Strawberry Flavour INH | 0.375 |
| Colloidal silicon dioxide | 0.75 |
| NF | |
| Lubrication | |
| Magnesium Stearate NF | 0.375 |
| Total weight of tablet | 100.00 |

Manufacturing Procedure:
1. Bedaquiline fumarate, piperine, microcrystalline cellulose, lactose monohydrate croscarmellose sodium and corn starch were weighed, sifted and blended.
2. Hypromellose and polysorbate 20 were added to purified water until dissolved.
3. The blend of step 1 was granulated with solution of step 2.
4. The granules of step 3 were granulated to suitable size.
5. Crospovidone, aspartame, strawberry flavour and colloidal silicon dioxide and magnesium stearate were sifted and blended with granules of step 5.
6. The blend was compressed to prepare core tablets.

### Example 4

Bedaquiline and Piperine oral powder for suspension

TABLE 4

| Ingredient | Quantity (%) |
|---|---|
| Blending | |
| Bedaquiline fumarate | 20.14 |
| Piperine | 3.33 |
| Microcrystalline | 8.35 |
| cellulose | |
| Lactose monohydrate | 24.16 |
| Croscarmellose sodium | 2.00 |
| Corn starch | 3.33 |
| Binder | |
| Hypromellose | 2.00 |
| Polysorbate 20 | 0.33 |
| Purified water | — |
| Blending | |
| Sorbitol powder | 27.08 |
| Xanthum gum | 1.50 |
| Monosodium citrate | 5.00 |
| (anhydrous) | |
| Sodium benzoate | 0.25 |
| Cream caramel flavour | 1.00 |
| Sodium saccharine | 1.00 |
| Titanium dioxide | 0.50 |
| Total fill weight of | 100.00 |
| sachet | |

Manufacturing Procedure:
1. Bedaquiline fumarate, piperine, microcrystalline cellulose, lactose monohydrate croscarmellose sodium and corn starch were weighed, sifted and blended.
2. Hypromellose and polysorbate 20 were added to purified water until dissolved.
3. The blend of step 1 was granulated with solution of step 2.
4. Sorbitol powder, xanthum gum, monosodium citrate, sodium benzoate, cream caramel flavour, sodium saccharine and titanium dioxide were sifted and blended with above blend of step 3.
5. The blend was filled in sachets on a suitable filling machine to prepare equal dosed sachets.

### Example 5

Delamanid and Piperine film coated tablets

TABLE 5

| Ingredient | Quantity (%) |
|---|---|
| Blending | |
| Delamanid | 16.12 |
| Piperine | 6.45 |
| Microcrystalline | 27.90 |
| cellulose | |
| Lactose monohydrate | 32.25 |
| Sodium starch glycolate | 3.87 |
| (Type A) | |
| Binder | |
| Povidone | 3.87 |
| all-rac-α-Tocopherol | 0.96 |
| Purified water | — |
| Blending | |
| Croscarmellose calcium | 3.87 |
| Colloidal silicon dioxide | 0.96 |
| Lubrication | |
| Magnesium Stearate | 0.48 |
| Total weight of Core | 100.00 |
| tablet | |
| Coating | |
| Opadry Yellow | 3.22 |
| Purified water | — |

Manufacturing Procedure:
1. Delamanid, piperine, microcrystalline cellulose, lactose monohydrate and sodium starch glycolate were weighed, sifted and blended.
2. Povidone was added to some quantity of water. all-rac-a-Tocopherol in warm water. Mixed the two solutions.
3. Granulated the mix of step 1 with solution of step 2.
4. The granules were dried and sized.
5. Croscarmellose calcium, colloidal silicon dioxide and magnesium stearate were sifted and blended with granules of step 4.
6. Compressed the blend of step 5 to prepare core tablets.
7. The core tablets were coated with Opadry Yellow INH.

### Example 6

### Delamanid and Piperine Capsules

TABLE 6

| Ingredient | Quantity (%) |
|---|---|
| Blending | |
| Delamanid | 16.66 |
| Piperine | 6.66 |
| Microcrystalline | 28.83 |
| cellulose | |
| Lactose monohydrate | 33.33 |
| Sodium starch glycolate | 4.00 |
| (Type A) | |
| Binder | |
| Povidone USP | 4.00 |
| all-rac-α-Tocopherol NF | 1.00 |
| Purified water | — |
| Blending | |
| Croscarmellose calcium | 4.00 |
| Colloidal silicon dioxide | 1.00 |
| Lubrication | |
| Magnesium stearate | 0.50 |
| Total fill weight | 100.00 |
| Capsule filling | |
| Empty hard gelatine | 95.00 |
| capsules shell size 0 | |
| Total weight of capsule | — |

Manufacturing Procedure:
1. Delamanid, piperine, microcrystalline cellulose, lactose monohydrate and Sodium starch glycolate weighed, sifted and blended.
2. Povidone was added to some quantity of water. all-rac-a-Tocopherol in warm water. Mixed the two solutions.
3. Granulated the mix of step 1 with solution of step 2.
4. The granules were dried and sized.
5. Croscarmellose calcium, colloidal silicon dioxide and magnesium stearate were sifted and blended with granules of step 4.
6. Filled the blend of step 5 on a suitable capsule filling machine to prepare capsules.

### Example 7

Delamanid and Piperine oral disintegrating tablets

TABLE 7

| Ingredient | Quantity (%) |
|---|---|
| Blending | |
| Delamanid | 16.66 |
| Piperine | 6.66 |
| Microcrystalline | 25.33 |
| cellulose | |
| Lactose monohydrate | 33.33 |
| Crospovidone | 4.00 |
| Binder | |
| Povidone USP | 4.00 |
| all-rac-α-Tocopherol NF | 1.00 |
| Purified water | — |
| Blending | |
| Crospovidone | 5.00 |
| Aspartame | 1.00 |
| Strawberry Flavour | 0.50 |
| Colloidal silicon dioxide | 1.00 |
| Lubrication | |
| Magnesium stearate | 0.50 |
| Total weight of tablet | 100.00 |

Manufacturing Procedure:
1. Delamanid, piperine, microcrystalline cellulose, lactose monohydrate and crospovidone were sifted and blended.
2. Povidone was added to some quantity of water. all-rac-a-Tocopherol in warm water. Mixed the two solutions.
3. Granulated the mix of step 1 with solution of step 2.
4. The granules were dried and sized.
5. Crospovidone, aspartame, strawberry flavour and colloidal silicon dioxide and magnesium stearate were added to blend of step 4.
6. Compressed the blend of step 5 to prepare core tablets.

### Example 8

Delamanid and Piperine oral powder for suspension

TABLE 8

| Ingredient | Quantity (%) |
|---|---|
| Blending | |
| Delamanid | 20.14 |
| Piperine | 3.33 |
| Microcrystalline | 8.35 |
| cellulose | |
| Lactose monohydrate | 24.16 |
| Croscarmellose sodium | 2.00 |
| Binder | |
| Povidone USP | 2.40 |
| all-rac-α-Tocopherol NF | 0.60 |
| Purified water | — |
| Blending | |
| Sorbitol powder | 35.15 |
| Xanthum gum | 1.50 |
| Monosodium citrate | 5.00 |
| (anhydrous) | |
| Sodium benzoate | 0.25 |
| Cream caramel flavour | 1.00 |
| Sodium saccharine | 1.00 |
| Titanium dioxide | 0.50 |
| Total fill weight of | 100.00 |
| sachet | |

Manufacturing Procedure:
1. Delamanid, piperine, microcrystalline cellulose, lactose monohydrate and crospovidone were sifted and blended.
2. Povidone was added to some quantity of water. all-rac-α-Tocopherol in warm water. Mixed the two solutions.
3. Granulated the mix of step 1 with solution of step 2.
4. The granules were dried and sized.
5. Sorbitol powder, xanthum gum, monosodium citrate, sodium benzoate, cream caramel flavour, sodium saccharine and titanium dioxide were blended and added to blend of step 4.
6. The blend of step 5 was filled in sachets on a suitable filling machine to prepare equal dosed sachets.

In order that this invention be more fully understood, the following preparative and testing methods are set forth. These methods are for the purpose of illustration only and are not to be construed as limiting the scope of the invention in any way.

### Material

Digoxin (known P-gp substrate), Bedaquiline, HBSS buffer, MES hydrate, HEPES powder, Fetal bovine serum (FBS), Minimum essential medium (MEM), Lucifer yellow, Piperine (P-gp inhibitor), Ketoconazole (P-gp inhibitor)

### Method

1. Caco-2 cell culture

Caco-2 cells were cultured in MEM media with 10% serum and seeded at a density of 75000 cells per mL and cultured for 21 days in a 24-well trans-well plate at 37° C., 5% CO₂ The monolayer integrity was checked intermittently (Day 0-21) using Trans Epithelial Electric Resistance (TEER). Cells were treated with drugs as follows:

Assay protocol

For A-B, 400 μL samples were added to the wells as per the plate setup to the apical side in duplicates with 800 μL HBSS pH 7.4 in the basal wells. Samples were collected at 60, 90 and 120 minutes from the basal side. Mass balance samples at 0 and 120 minutes were collected from the apical side.

For B-A, 800 μL of the respective dilutions were added to the basal side in duplicates with 400 μL HBSS pH 7.4 in the apical wells. Samples were collected at 60, 90 and 120 minutes from the apical side. Mass balance samples at 0 and 120 minutes were collected from the basal side.

The sample were analyzed on LCMS-MS.

At the end of the experiment the monolayer integrity was checked using and Lucifer yellow, calculating the % rejection of Lucifer yellow by incubating cells with 100 μg/mL Lucifer.

Papp was calculated as follows:

The apparent permeability (Papp) in units per second can be calculated by using the following equation,

For single point method:

Papp=(V/(T*A))*(C0//Ct)

For multi-point method:

Papp=(dQ/dt)/(A*C0)

% Mass balance=100-[CR120*VR+CD120*VD/C0*VD]

For Lucifer yellow,

% Lucifer Yellow Passage=[RFU (test)−RFU (blank)/RFU (equilibrium)−RFU (blank)]*100

Permeability classification:

| Permeability | Papp (nm/s) |
|---|---|
| Low | <50 |
| Moderate | 50-200 |
| High | >200 |

Efflux ratio=Papp B−A/Papp A−B

Efflux ratio≥2 indicates that the drug is a P-gp substrate

Results:

Bedaquiline is a known P-gp substrate and in presence of piperine improves the permeability.

Conclusions:

It is concluded that absorption of Bedaquiline is increased with piperine by decreasing the efflux ratio.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the ail, and such modifications and variations are considered to be falling within the scope of the nention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

We claims:

1. A pharmaceutical formulation comprising a therapeutically effective amount of at least one antituberculosis drug or its pharmaceutically acceptable salts or derivatives thereof, at least one bioenhancer or its derivative thereof and optionally one or more pharmaceutically acceptable excipients.

2. The pharmaceutical formulation of claim 1, wherein the antituberculosis drug is selected from bedaquiline, delamanid, pretomanid and the combinations thereof.

3. The pharmaceutical formulation of claim 1 wherein the antituberculosis drug is bedaquiline or its pharmaceutically acceptable salts or derivatives thereof.

4. The pharmaceutical formulation of claim 3, wherein the amount of bedaquiline is 10% to 40% w/w of the total formulation.

5. The pharmaceutical formulation of claim 1, wherein the bioenhancer is selected from piperine, garlic, Carum carvi, Currinum cyrrinurn lysergol, naringin, quercetin, niaziridin, glycyrrhizin, stevia, cow urine, distillate ginger, or any combination thereof.

6. The pharmaceutical formulation of claim 5, wherein bioenhancer is selected from synthetically prepared piperine, extract from black pepper and extract from piper longum.

7. The pharmaceutical formulation of claim 6, wherein bioenhancer is selected from tetrahydropiperine, cis-piperine, transpiperine, cis-trans piperine, trans,cis-piperine, cis,cis-piperine, trans, transpiperine or a combination thereof.

8. The pharmaceutical formulation of claim 1, wherein the piperine is present at an amount from about 0.5 mg to about 400 mg in the formulation.

9. The pharmaceutical composition of claim 1, wherein the ratio of antituberculosis drug and piperine is from about 100:1 to about 1:1 by weight of the formulation.

10. The pharmaceutical composition of claim 1, wherein the composition is in the form of a tablet, mini-tablet, granules, sprinkles, capsules, sachets, powders, pellets, disintegrating tablets, dispersible tablets, solution, suspension, emulsion, lyophilized powder or in the form of a kit.

11. The pharmaceutical formulation of claim 1 further comprises of additional anti-HIV drugs selected from zidovudine or AZT, didanosine, stavudine, lamivudine, zalcitabine, tenofovir disoproxil fumarate, tenofovir alafenamide, emtricitabine, efavirenz, doravarine, lamivudine, zidovudine, didanosine, stavudine, abacavir, etravirine, delavirdine, nevirapine or their salt, solvate, esters, derivatives, hydrate, enantiomer, polymorph prodrugs, tautomers, isomers, anhydrates or mixtures thereof.

12. A method of enhancing the bioavailability of bedaquiline from about 10% to about 100%, the method comprising administering a combination product comprising therapeutically effective amount of bedaquiline or its pharmaceutically acceptable salts, derivatives thereof and piperine or its derivative thereof simultaneously, separately or sequentially to a patient in need thereof.

13. A method of decreasing the dose of bedaquiline from about from about 5% to about 95%, the method comprising administering a combination product comprising therapeutically effective amount of bedaquiline or its pharmaceutically acceptable salts or derivatives thereof, piperine or its pharmaceutically acceptable derivatives thereof simultaneously, separately or sequentially to a patient in need thereof.

14. A method of treating diseases caused by mycobacterium tuberculosis in a patient in need of treatment thereof, the method comprising administering a pharmaceutical composition comprising a therapeutically effective amount of bedaquiline or its pharmaceutically acceptable salts or derivatives thereof; piperine or its pharmaceutically acceptable derivative thereof; and
optionally one or more pharmaceutically acceptable excipients.

15. The method according to claim 14, wherein the diseases caused by mycobacterium tuberculosis are treatments of MDR-TB, XDRTB, and TDR-TB.

16. A kit comprising therapeutically effective amount of bedaquiline or its pharmaceutically acceptable salts or derivatives thereof in an amount effective and piperine or its pharmaceutically acceptable derivative thereof to treat diseases caused by mycobacterium tuberculosis.

17. The kit of claim 16, wherein the bedaquiline or its pharmaceutically acceptable salts or derivatives thereof; piperine or its pharmaceutically acceptable derivative thereof are present in same or separate formulation for simultaneously, separately or sequentially to a patient in need thereof.
